(19) United States
(12) Patent Application Publication
(10) Pub. No.: US 2018/0364343 A1
(43) Pub. Date: Dec. 20, 2018

(54) ULTRASOUND IMAGING RECEIVER CIRCUIT

(71) Applicant: Yahui BAI, Hong Kong (HK)

(72) Inventor: Yahui BAI, Hong Kong (HK)

(73) Assignee: YAHUIKANG LIMITED, Hong Kong (HK)

(21) Appl. No.: 16/108,089

(22) Filed: Aug. 21, 2018

(51) Int. Cl. G01S 7/52; A61B 8/08; G01S 15/89

(52) U.S. Cl. CPC G01S 7/52028; A61B 8/48; G01S 15/8934; G01S 7/5208; G01S 7/52033

(57) ABSTRACT

An ultrasound imaging receiver circuit includes a plurality of amplifiers configured to receive input signals from a plurality of ultrasound transducer channels respectively; a plurality of ADCs connected with the amplifiers respectively; a switching circuit connected with the ADCs; an image signal processor connected with the switching circuit; and a control signal generator. Each amplifier includes a first input port configured for receiving one input signal from an ultrasound transducer channel, a second input port connected to the ground and an output port connected to one ADC, the first input port being connected to the output port through a first switch. The control signal generator is configured to compare the input signal with a first preset threshold and a second preset threshold and output control signals controlling the first switch and the second switch based on the comparisons.

### FIELD OF THE PATENT APPLICATION

The present patent application generally relates to medical electronics and more specifically to an ultrasound imaging receiver circuit.

### BACKGROUND

Ultrasound probes of an ultrasound imaging system typically include one or more ultrasound transducers which sense ultrasound signals and produce corresponding electrical signals. The electrical signals are processed in the analog or digital domain and the processed electrical signals are then used to generate ultrasound images.

FIG. 1 is a schematic circuit diagram of a conventional ultrasound imaging receiver circuit. Referring to FIG. 1, the ultrasound imaging receiver circuit includes a plurality of amplifiers 103 configured to receive input signals (IN1, IN2 and IN3 as shown in FIG. 1) from a plurality of ultrasound transducer channels respectively, a switching circuit 105 connected with the amplifiers 103, an ADC 107 connected with the switching circuit 105, and an image signal processor 109 connected with the ADC 107. The system requires only one ADC 107 for handling signals coming from all ultrasound transducer channels, which helps to reduce power consumption. However, because the digital conversion is executed only after the input signals IN1, IN2 and IN3 are amplified by the amplifier 103 and selected by the switching circuit 105, finer adjustments and digital manipulation of the input signals cannot be implemented with this architecture.

Some digital beamforming imaging systems have been proposed in recent years but their relatively high power consumption and relatively low dynamic range are the key bottlenecks for such systems.

### SUMMARY

The present patent application is directed to an ultrasound imaging receiver circuit. In one aspect, the ultrasound imaging receiver circuit includes a plurality of amplifiers configured to receive input signals from a plurality of ultrasound transducer channels respectively; a plurality of ADCs connected with the amplifiers respectively; a switching circuit connected with the ADCs; an image signal processor connected with the switching circuit; and a control signal generator. Each amplifier includes a first input port configured for receiving one input signal from an ultrasound transducer channel, a second input port connected to the ground and an output port connected to one ADC, the first input port being connected to the output port through a first switch. The amplifier further includes a first bias port connected to a power supply voltage and a second bias port connected to the ground through a second switch. The control signal generator is electrically connected with the first switch and the second switch and including an input port configured for receiving the input signal; a first output port configured for outputting a first control signal controlling the first switch; and a second output port configured for outputting a second control signal controlling the second switch. The control signal generator is configured to compare the input signal with a first preset threshold; if the input signal is greater than the first preset threshold, the control signal generator is configured to output a first control signal that turns on the first switch through the first output port and a second control signal that turns off the second switch through the second output port; and the control signal generator is configured to compare the input signal with a second preset threshold; if the input signal is less than the second preset threshold, the control signal generator is configured to output a first control signal that turns off the first switch through the first output port, and a second control signal that turns on the second switch through the second output port.

The control signal generator may include at least a comparator. The ADC may be reconfigurable and when the input signal is greater than the first preset threshold, the gain of the ADC may be configured to increase with attenuation of the input signal so that the SNR of the signal output from the ADC may be roughly constant.

The gain of the amplifier may be tunable and adjusted to be increasing with the attenuation of the input signal so that when the input signal is less than the second preset threshold the SNR of the signal output from the ADC may be roughly constant.

In another aspect, the present patent application provides an ultrasound imaging receiver circuit. The ultrasound imaging receiver circuit includes a plurality of amplifiers configured to receive input signals from a plurality of ultrasound transducer channels respectively; a plurality of ADCs connected with the amplifiers respectively; a switching circuit connected with the ADCs; an image signal processor connected with the switching circuit; and a control signal generator. Each amplifier includes a first input port configured for receiving one input signal from an ultrasound transducer channel, a second input port connected to the ground and an output port connected to one ADC, the first input port being connected to the output port through a first switch. The amplifier further includes a first bias port connected to a power supply voltage and a second bias port connected to the ground through a second switch. The control signal generator is connected with the first switch and the second switch and comprising an input port configured for receiving the input signal; a first output port configured for outputting a first control signal controlling the first switch; and a second output port configured for outputting a second control signal controlling the second switch.

The control signal generator may be configured to compare the input signal with a first preset threshold; if the input signal is greater than the first preset threshold, the control signal generator may be configured to output a first control signal that turns on the first switch through the first output port and a second control signal that turns off the second switch through the second output port.

The control signal generator may be configured to compare the input signal with a second preset threshold; if the input signal is less than the second preset threshold, the control signal generator may be configured to output a first control signal that turns off the first switch through the first output port, and a second control signal that turns on the second switch through the second output port. The control signal generator may include at least a comparator.

The ADC may be reconfigurable and when the input signal is greater than the first preset threshold, the gain of the ADC may be configured to increase with attenuation of the input signal so that the SNR of the signal output from the ADC may be roughly constant.

The gain of the amplifier may be tunable and adjusted to be increasing with the attenuation of the input signal so that when the input signal is less than the second preset threshold the SNR of the signal output from the ADC may be roughly constant.

In yet another aspect, the ultrasound imaging receiver circuit includes a plurality of amplifiers configured to receive input signals from a plurality of ultrasound transducer channels respectively; a plurality of ADCs connected with the amplifiers respectively; a switching circuit connected with the ADCs; an image signal processor connected with the switching circuit; and a control signal generator. Each amplifier includes a first input port configured for receiving one input signal from an ultrasound transducer channel, a second input port connected to the ground and an output port connected to one ADC, the first input port being connected to the output port through a first switch. The amplifier further includes a first bias port connected to a power supply voltage and a second bias port connected to the ground through a second switch. The control signal generator is connected with the first switch and the second switch and including an input port configured for receiving the input signal; a first output port configured for outputting a first control signal controlling the first switch; and a second output port configured for outputting a second control signal controlling the second switch. The control signal generator is configured to compare the input signal with a first preset threshold and a second preset threshold and output control signals controlling the first switch and the second switch based on the comparisons.

If the input signal is greater than the first preset threshold, the control signal generator may be configured to output a first control signal that turns on the first switch through the first output port and a second control signal that turns off the second switch through the second output port.

If the input signal is less than the second preset threshold, the control signal generator may be configured to output a first control signal that turns off the first switch through the first output port, and a second control signal that turns on the second switch through the second output port.

The control signal generator may include at least a comparator. The ADC may be reconfigurable and when the input signal is greater than the first preset threshold, the gain of the ADC may be configured to increase with attenuation of the input signal so that the SNR of the signal output from the ADC may be roughly constant.

The gain of the amplifier may be tunable and adjusted to be increasing with the attenuation of the input signal so that when the input signal is less than the second preset threshold the SNR of the signal output from the ADC may be roughly constant.

### BRIEF DESCRIPTION OF THE DRAWINGS

FIG. 1 is a schematic circuit diagram of a conventional ultrasound imaging receiver circuit.

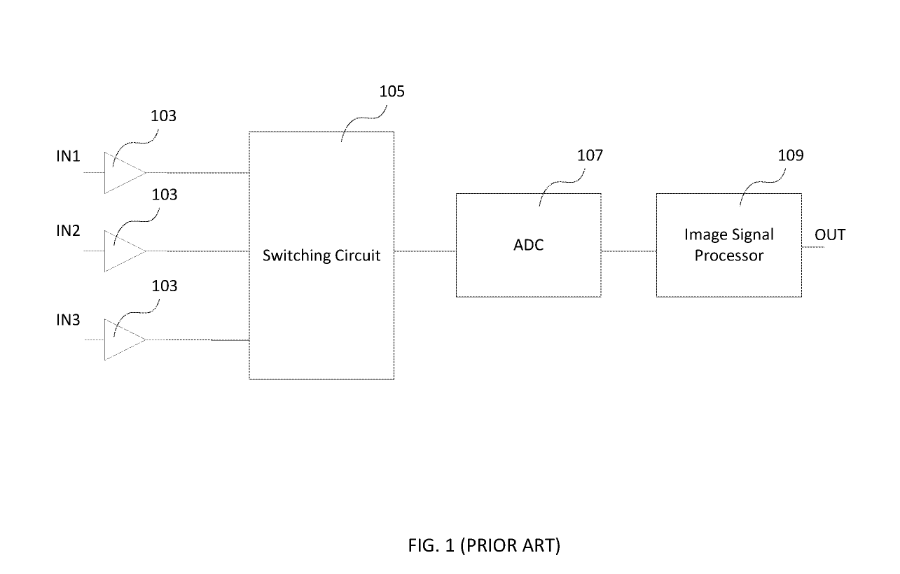

FIG. 2 is a schematic circuit diagram of an ultrasound imaging receiver circuit in accordance with an embodiment of the present patent application.

FIG. 3 is a schematic circuit diagram illustrating the configuration of an amplifier of the ultrasound imaging receiver circuit as depicted in FIG. 2.

### DETAILED DESCRIPTION

Reference will now be made in detail to a preferred embodiment of the ultrasound imaging receiver circuit disclosed in the present patent application, examples of which are also provided in the following description. Exemplary embodiments of the ultrasound imaging receiver circuit disclosed in the present patent application are described in detail, although it will be apparent to those skilled in the relevant art that some features that are not particularly important to an understanding of the ultrasound imaging receiver circuit may not be shown for the sake of clarity.

Furthermore, it should be understood that the ultrasound imaging receiver circuit disclosed in the present patent application is not limited to the precise embodiments described below and that various changes and modifications thereof may be effected by one skilled in the art without departing from the spirit or scope of the protection. For example, elements and/or features of different illustrative embodiments may be combined with each other and/or substituted for each other within the scope of this disclosure.

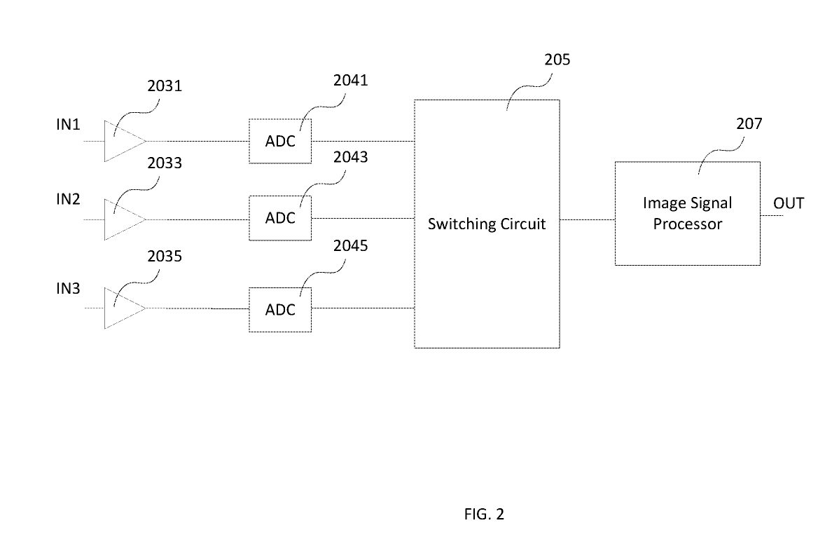

FIG. 2 is a schematic circuit diagram of an ultrasound imaging receiver circuit in accordance with an embodiment of the present patent application. Referring to FIG. 2, the ultrasound imaging receiver circuit includes a plurality of amplifiers (2031, 2033 and 2035) configured to receive input signals (IN1, IN2 and IN3 as shown in FIG. 2) from a plurality of ultrasound transducer channels respectively; a plurality of ADCs (2041, 2043 and 2045) connected with the amplifiers (2031, 2033 and 2035) respectively; a switching circuit 205 connected with the ADCs (2041, 2043 and 2045); and an image signal processor 207 connected with the switching circuit 205.

It is noted that in the above system each ultrasound transducer channel has a dedicated ADC (2041, 2043 or 2045). As a result, finer adjustments and digital manipulation of the input signal from each ultrasonic transducer channel can be implemented.

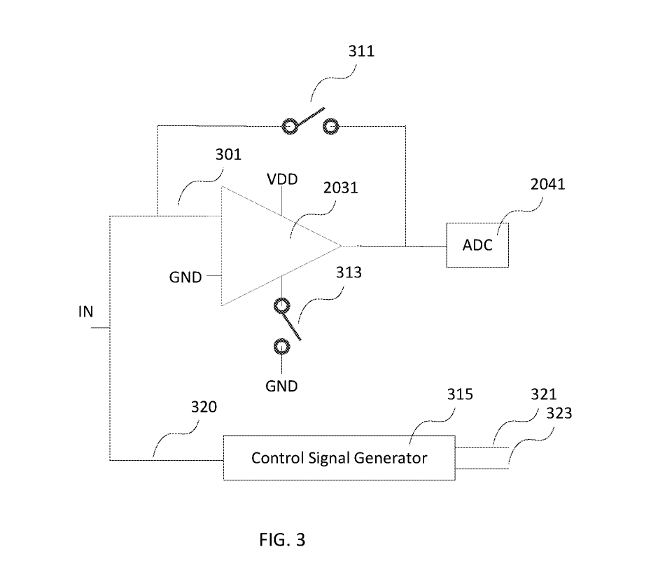

FIG. 3 is a schematic circuit diagram illustrating the configuration of the amplifier 2031 as depicted in FIG. 2. Referring to FIG. 3, the amplifier 2031 includes a first input port 301 configured for receiving the input signal IN1, a second input port connected to the ground (GND) and an output port connected to the corresponding ADC 2041. The first input port 301 is also connected to the output through a first switch 311. The amplifier 2031 further includes a first bias port connected to a power supply voltage VDD and a second bias port connected to the ground through a second switch 313.

The ultrasound imaging receiver circuit further includes a control signal generator 315 electrically connected with the first switch 311 and the second switch 313 (the connections are not shown in FIG. 3). The control signal generator 315 includes an input port 320 configured for receiving the input signal IN1; a first output port 321 configured for outputting a first control signal controlling the first switch 311; and a second output port 323 configured for outputting a second control signal controlling the second switch 313. In this embodiment, the control signal generator 315 includes at least a comparator.

At the beginning of each signal cycle, the input signal IN1, for example, is relatively strong. The control signal generator 315 is configured to compare the input signal IN1 with a first preset threshold T1. If the input signal IN1 is greater than the first preset threshold T1, the control signal generator 315 is configured to output an output signal (i.e. a first control signal) “1” through the first output port 321, which turns on the first switch 311, and an output signal (i.e. a second control signal) “0” through the second output port 323, which turns off the second switch 313. Under this condition, referring to FIG. 2 and FIG. 3, the amplifier 2031 is bypassed and the input signal IN1 is directly fed to the ADC 2041. Since the second switch 313 is turned off, the amplifier 2031 is turned off and not consuming any power.

Preferably, in this embodiment, the ADC 2041 is reconfigurable and the gain of the ADC 2041 is configured to increase with the attenuation of the input signal IN1 so that the SNR of the signal output from the ADC 2041 is roughly constant. As a result, the dynamic range of the system is kept constant without being narrowed by the attenuation of the input signal IN1.

As time passes by, the input signal IN1 attenuates and becomes weaker and weaker. The control signal generator 315 is configured to compare the input signal IN1 with a second preset threshold T2. If the input signal IN1 is less than the second preset threshold T2, the control signal generator 315 is configured to output an output signal (i.e. a first control signal) “0” through the first output port 321, which turns off the first switch 311, and an output signal (i.e. a second control signal) “1” through the second output port 323, which turns on the second switch 313. Under this condition, referring to FIG. 2 and FIG. 3, the amplifier 2031 is working and the input signal IN1 is first amplified by the corresponding amplifier 2031 and then fed to the ADC 2041.

Preferably, the gain of the amplifier 2031 is tunable and adjusted to be increasing with the attenuation of the input signal IN1 so that the SNR of the signal output from the ADC 2041 is roughly constant. As a result, the dynamic range of the system is kept constant without being narrowed by the attenuation of the input signal IN1.

Although the amplifier 2031 and the ADC 2041, which correspond to one ultrasound transducer channel, are described above as an example, as all ultrasound transducer channels are identical, it is understood that the configuration and the working principle of the amplifiers 2033 and 2035 are similar to those of the amplifier 2031, while the configuration and the working principle of the ADCs 2043 and 2045 are similar to those of the ADC 2041.

It is further understood that, in an alternative embodiment, the number of ultrasound transducer channels may be more or less than 3.

In the above embodiments, since each ultrasound transducer channel has a dedicated ADC (2041, 2043 or 2045), finer adjustments and digital manipulation of the input signals from ultrasound transducer channels can be implemented with the ultrasound imaging receiver circuit. When the input signal IN1 (or IN2, IN3, . . . ) is relatively strong and greater than the first preset threshold, the amplifier 2031 (or 2033, 2035 . . . ) is turned off and not consuming any power, and therefore the average power consumption of the system is relatively low. With the conditional combinational use of the amplifier 2031 (or 2033, 2035 . . . ) and the ADC 2041 (or 2043, 2045, . . . ), the SNR of the output signal of the system is kept roughly constant. As a result, the dynamic range of the system is kept roughly constant without being narrowed by the attenuation of the input signal IN1 (or IN2, IN3,).

Therefore, the ultrasound imaging receiver circuit offers the possibility of finer adjustments and digital manipulation of the input signals while enjoying the benefits of low power consumption and wide dynamic range.

While the present patent application has been shown and described with particular references to a number of embodiments thereof, it should be noted that various other changes or modifications may be made without departing from the scope of the present invention.

What is claimed is:

1. An ultrasound imaging receiver circuit comprising:
a plurality of amplifiers configured to receive input signals from a plurality of ultrasound transducer channels respectively;
a plurality of ADCs connected with the amplifiers respectively;
a switching circuit connected with the ADCs;
an image signal processor connected with the switching circuit and a control signal generator; wherein:
each amplifier comprises a first input port configured for receiving one input signal from an ultrasound transducer channel, a second input port connected to the ground and an output port connected to one ADC, the first input port being connected to the output port through a first switch;
the amplifier further comprises a first bias port connected to a power supply voltage and a second bias port connected to the ground through a second switch;
the control signal generator is electrically connected with the first switch and the second switch and comprising an input port configured for receiving the input signal; a first output port configured for outputting a first control signal controlling the first switch; and a second output port configured for outputting a second control signal controlling the second switch;
the control signal generator is configured to compare the input signal with a first preset threshold; if the input signal is greater than the first preset threshold, the control signal generator is configured to output a first control signal that turns on the first switch through the first output port and a second control signal that turns off the second switch through the second output port; and
the control signal generator is configured to compare the input signal with a second preset threshold; if the input signal is less than the second preset threshold, the control signal generator is configured to output a first control signal that turns off the first switch through the first output port, and a second control signal that turns on the second switch through the second output port.

2. The ultrasound imaging receiver circuit of claim 1, wherein the control signal generator comprises at least a comparator.

3. The ultrasound imaging receiver circuit of claim 1, wherein the ADC is reconfigurable and when the input signal is greater than the first preset threshold, the gain of the ADC is configured to increase with attenuation of the input signal so that the SNR of the signal output from the ADC is roughly constant.

4. The ultrasound imaging receiver circuit of claim 1, wherein the gain of the amplifier is tunable and adjusted to be increasing with the attenuation of the input signal so that when the input signal is less than the second preset threshold the SNR of the signal output from the ADC is roughly constant.

5. An ultrasound imaging receiver circuit comprising:
a plurality of amplifiers configured to receive input signals from a plurality of ultrasound transducer channels respectively;
a plurality of ADCs connected with the amplifiers respectively;
a switching circuit connected with the ADCs;
an image signal processor connected with the switching circuit and a control signal generator; wherein:
each amplifier comprises a first input port configured for receiving one input signal from an ultrasound transducer channel, a second input port connected to the ground and an output port connected to one ADC, the first input port being connected to the output port through a first switch;
the amplifier further comprises a first bias port connected to a power supply voltage and a second bias port connected to the ground through a second switch; and
the control signal generator is connected with the first switch and the second switch and comprising an input port configured for receiving the input signal; a first output port configured for outputting a first control signal controlling the first switch; and a second output port configured for outputting a second control signal controlling the second switch.

6. The ultrasound imaging receiver circuit of claim 5, wherein the control signal generator is configured to compare the input signal with a first preset threshold; if the input signal is greater than the first preset threshold, the control signal generator is configured to output a first control signal that turns on the first switch through the first output port and a second control signal that turns off the second switch through the second output port.

7. The ultrasound imaging receiver circuit of claim 5, wherein the control signal generator is configured to compare the input signal with a second preset threshold; if the input signal is less than the second preset threshold, the control signal generator is configured to output a first control signal that turns off the first switch through the first output port, and a second control signal that turns on the second switch through the second output port.

8. The ultrasound imaging receiver circuit of claim 5, wherein the control signal generator comprises at least a comparator.

9. The ultrasound imaging receiver circuit of claim 5, wherein the ADC is reconfigurable and when the input signal is greater than the first preset threshold, the gain of the ADC is configured to increase with attenuation of the input signal so that the SNR of the signal output from the ADC is roughly constant.

10. The ultrasound imaging receiver circuit of claim 5, wherein the gain of the amplifier is tunable and adjusted to be increasing with the attenuation of the input signal so that when the input signal is less than the second preset threshold the SNR of the signal output from the ADC is roughly constant.

11. An ultrasound imaging receiver circuit comprising:
a plurality of amplifiers configured to receive input signals from a plurality of ultrasound transducer channels respectively;
a plurality of ADCs connected with the amplifiers respectively;
a switching circuit connected with the ADCs;
an image signal processor connected with the switching circuit and
a control signal generator; wherein:
each amplifier comprises a first input port configured for receiving one input signal from an ultrasound transducer channel, a second input port connected to the ground and an output port connected to one ADC, the first input port being connected to the output port through a first switch;
the amplifier further comprises a first bias port connected to a power supply voltage and a second bias port connected to the ground through a second switch;
the control signal generator is connected with the first switch and the second switch and comprising an input port configured for receiving the input signal; a first output port configured for outputting a first control signal controlling the first switch; and a second output port configured for outputting a second control signal controlling the second switch; and
the control signal generator is configured to compare the input signal with a first preset threshold and a second preset threshold and output control signals controlling the first switch and the second switch based on the comparisons.

12. The ultrasound imaging receiver circuit of claim 11, wherein if the input signal is greater than the first preset threshold, the control signal generator is configured to output a first control signal that turns on the first switch through the first output port and a second control signal that turns off the second switch through the second output port.

13. The ultrasound imaging receiver circuit of claim 11, wherein if the input signal is less than the second preset threshold, the control signal generator is configured to output a first control signal that turns off the first switch through the first output port, and a second control signal that turns on the second switch through the second output port.

14. The ultrasound imaging receiver circuit of claim 11, wherein the control signal generator comprises at least a comparator.

15. The ultrasound imaging receiver circuit of claim 11, wherein the ADC is reconfigurable and when the input signal is greater than the first preset threshold, the gain of the ADC is configured to increase with attenuation of the input signal so that the SNR of the signal output from the ADC is roughly constant.

16. The ultrasound imaging receiver circuit of claim 11, wherein the gain of the amplifier is tunable and adjusted to be increasing with the attenuation of the input signal so that when the input signal is less than the second preset threshold the SNR of the signal output from the ADC is roughly constant.
